(19) United States
(12) Patent Application Publication
(10) Pub. No.: US 2026/0325101 A1
(43) Pub. Date: Oct. 22, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventors: Hidetoshi TAKAYAMA, Kanagawa (JP); Tomoki INOUE, Kanagawa (JP); Tetsurou EBATA, Kanagawa (JP)

(73) Assignee: FUJIFILM Corporation, Tokyo (JP)

(21) Appl. No.: 19/632,388

(22) Filed: Mar. 29, 2026

(30) Foreign Application Priority Data
Apr. 2, 2025 (JP) 2025-060993
Apr. 2, 2025 (JP) 2025-061038

(51) Int. Cl. A61B 8/08; A61B 5/00; A61B 5/01; A61B 5/0245; A61B 5/1455; A61B 8/02; A61B 8/04; A61B 8/06

(52) U.S. Cl. CPC A61B 8/0883; A61B 5/01; A61B 5/0245; A61B 5/1455; A61B 5/746; A61B 8/02; A61B 8/04; A61B 8/06

(57) ABSTRACT

Provided is an ultrasound diagnostic apparatus capable of smoothly carry out the examination of subject's cardiac function and a control method of the ultrasound diagnostic apparatus

An ultrasound diagnostic apparatus includes a first measurement unit that measures a left ventricular ejection fraction based on an ultrasound image in which the heart of a subject is imaged, a second measurement unit that measures a blood flow-related index related to blood flow based on the ultrasound image, and a measurement controller that controls the second measurement unit such that when the left ventricular ejection fraction measured by the first measurement unit is equal to or smaller than a predetermined threshold value, the blood flow-related index is measured by the second measurement unit.

### CROSS-REFERENCE TO RELATED APPLICATIONS

The present application claims priority under 35 U.S.C. § 119 to Japanese Patent Application No. 2025-060993, filed on Apr. 2, 2025 and Japanese Patent Application No. 2025-061038, filed on Apr. 2, 2025. The above application is hereby expressly incorporated by reference, in those entirety, into the present application.

### BACKGROUND OF THE INVENTION

### 1. Field of the Invention

The present invention relates to an ultrasound diagnostic apparatus for imaging the heart of a subject and a control method of the ultrasound diagnostic apparatus.

### 2. Description of the Related Art

An examination of cardiac function of a subject has been conventionally carried out by acquiring an ultrasound image that represents a tomographic plane of a subject's heart using a so-called ultrasound diagnostic apparatus and analyzing the acquired ultrasound image. For instance, WO 2012/023399 discloses measuring a so-called left ventricular ejection fraction and a so-called cardiac output as cardiac function of the subject using an ultrasound diagnostic apparatus.

### SUMMARY OF THE INVENTION

In general, it may be difficult to fully perceive pathological conditions about the subject's heat function based only on the left ventricular ejection fraction. Hence, it is considered that the cardiac output in addition to the left ventricular ejection fraction is measured, but a user with low skills in the cardiac function examination sometimes cannot decide that it is necessary to measure the cardiac output. Moreover, a user is usually required to manually operate an ultrasound diagnostic apparatus to change from the measurement of the left ventricular ejection fraction to the measurement of the cardiac output, and it may be thus difficult to smoothly proceed with the examination.

The present invention has been made to solve the above-described conventional problem and has an object to provide an ultrasound diagnostic apparatus capable of smoothly carry out the examination of subject's cardiac function and a control method of the ultrasound diagnostic apparatus.

With the following configuration, the above-described object can be achieved.
[1] An ultrasound diagnostic apparatus comprising:
a first measurement unit that measures a left ventricular ejection fraction based on an ultrasound image in which a heart of a subject is imaged;
a second measurement unit that measures a blood flow-related index related to blood flow based on the ultrasound image; and
a measurement controller that controls the second measurement unit such that when the left ventricular ejection fraction measured by the first measurement unit is equal to or smaller than a predetermined threshold value, the blood flow-related index is measured by the second measurement unit.
[2] The ultrasound diagnostic apparatus according to [1], wherein the second measurement unit measures a blood flow velocity-time integral value and acquires a stroke volume as the blood flow-related index based on the blood flow velocity-time integral value measured and a left ventricular outflow tract diameter.
[3] The ultrasound diagnostic apparatus according to [2], wherein the second measurement unit measures the left ventricular outflow tract diameter and acquires the stroke volume based on the left ventricular outflow tract diameter measured and the blood flow velocity-time integral value measured.
[4] The ultrasound diagnostic apparatus according to [2] or [3], comprising a time integral value determination unit that determines whether a measurement result of the blood flow velocity-time integral value acquired by the second measurement unit falls within a predetermined acceptable range of time integral value,
wherein the second measurement unit acquires the stroke volume based on a specified value of the left ventricular outflow tract diameter and the blood flow velocity-time integral value measured when the time integral value determination unit determines that the blood flow velocity-time integral value falls within the acceptable range of the time integral value.
[5] The ultrasound diagnostic apparatus according to any one of [2] to [4], comprising a heart rate measurement unit that measures a heart rate of the subject.
[6] The ultrasound diagnostic apparatus according to [5], wherein the second measurement unit calculates a cardiac output based on the stroke volume acquired and the heart rate measured by the heart rate measurement unit.
[7] The ultrasound diagnostic apparatus according to [5] or [6], comprising a notification unit that notifies a user when the heart rate measured by the heart rate measurement unit fluctuates beyond a predetermined fluctuation range of a heart rate.
[8] The ultrasound diagnostic apparatus according to [1], wherein the second measurement unit acquires a blood flow velocity-time integral value as the blood flow-related index.
[9] The ultrasound diagnostic apparatus according to any one of [1] to [8], wherein the measurement controller has the predetermined threshold value that is set based on past statistical data.
[10] The ultrasound diagnostic apparatus according to any one of [1] to [8], wherein the measurement controller has the predetermined threshold value that is individually set depending on the subject.
[11] The ultrasound diagnostic apparatus according to [10], wherein the measurement controller has the predetermined threshold value that is dynamically set based on an evaluation result of fluctuation in at least one of blood pressure, heart rate, or peripheral perfusion index of the subject.
[12] The ultrasound diagnostic apparatus according to any one of [1] to [11], comprising a third measurement unit that measures a hemodynamic index representing hemodynamics of the subject,
wherein the measurement controller controls the second measurement unit such that when the left ventricular ejection fraction measured by the first measurement unit is equal to or larger than a predetermined threshold value and the hemodynamic index measured by the third measurement unit shows abnormality, the blood flow-related index is measured by the second measurement unit.
[13] The ultrasound diagnostic apparatus according to [12], wherein the third measurement unit is a non-invasive sphygmomanometer that measures blood pressure as the hemodynamic index.
[14] The ultrasound diagnostic apparatus according to [12], wherein the third measurement unit is an electrocardiogram monitor that measures a heart rate as the hemodynamic index.
[15] The ultrasound diagnostic apparatus according to [12], wherein the third measurement unit is a pulse oximeter that measures peripheral oxygen saturation as the hemodynamic index.
[16] The ultrasound diagnostic apparatus according to [12], wherein the third measurement unit is a lactate meter that measures a lactate level as the hemodynamic index.
[17] The ultrasound diagnostic apparatus according to [12], wherein the third measurement unit is a thermometer that measures peripheral skin temperature as the hemodynamic index.
[18] The ultrasound diagnostic apparatus according to any one of [2] to [4], comprising a third measurement unit that measures a hemodynamic index representing hemodynamics of the subject,
wherein the measurement controller controls the second measurement unit such that when the left ventricular ejection fraction measured by the first measurement unit is equal to or larger than the predetermined threshold value and the hemodynamic index measured by the third measurement unit shows abnormality, the blood flow-related index is measured by the second measurement unit,
the third measurement unit is an electrocardiogram monitor that measures a heart rate as the hemodynamic index, and
the second measurement unit calculates a cardiac output based on the stroke volume acquired and the heart rate measured by the electrocardiogram monitor.
[19] A method of controlling an ultrasound diagnostic apparatus, the method comprising:
measuring a left ventricular ejection fraction based on an ultrasound image in which a heart of a subject is imaged; and
measuring a blood flow-related index related to blood flow based on the ultrasound image when the left ventricular ejection fraction is equal to or smaller than a predetermined threshold value.

An ultrasound diagnostic apparatus according to an aspect of the present invention comprises: a first measurement unit that measures a left ventricular ejection fraction based on an ultrasound image in which a heart of a subject is imaged; a second measurement unit that measures a blood flow-related index related to blood flow based on the ultrasound image; and a measurement controller that controls the second measurement unit such that when the left ventricular ejection fraction measured by the first measurement unit is equal to or smaller than a predetermined threshold value, the blood flow-related index is measured by the second measurement unit. Therefore, it is possible to smoothly carry out the examination of subject's cardiac function.

### BRIEF DESCRIPTION OF THE DRAWINGS

FIG. 1 is a block diagram showing a configuration of an ultrasound diagnostic apparatus according to Embodiment 1 of the present invention.

FIG. 2 is a block diagram showing an internal configuration of a transmission/reception circuit in Embodiment 1 of the present invention.

FIG. 3 is a block diagram showing an internal configuration of an image generation unit in Embodiment 1 of the present invention.

FIG. 4 is a diagram showing an example of an ultrasound image showing an apical four-chamber view.

FIG. 5 is a diagram showing an example of an ultrasound image showing a parasternal left ventricular long-axis view.

FIG. 6 is a diagram showing an example of an ultrasound image showing an apical five-chamber view.

FIG. 7 is a block diagram showing an internal configuration of a second measurement unit in Embodiment 1 of the present invention.

FIG. 8 is a diagram showing an example of a measurement line disposed in the ultrasound image showing the parasternal left ventricular long-axis view.

FIG. 9 is a diagram showing an example of a Doppler gate disposed in the ultrasound image showing the apical five-chamber view.

FIG. 10 is a diagram showing an exemplary indication of an invitation for measurement of a stroke volume.

FIG. 11 is a flow chart showing an operation of the ultrasound diagnostic apparatus according to Embodiment 1 of the present invention.

FIG. 12 is a block diagram showing an internal configuration of a second measurement unit in a first modification of Embodiment 1 of the present invention.

FIG. 13 is a block diagram showing an internal configuration of a second measurement unit in a second modification of Embodiment 1 of the present invention.

FIG. 14 is a block diagram showing a configuration of an ultrasound diagnostic apparatus according to Embodiment 2 of the present invention.

FIG. 15 is a block diagram showing an internal configuration of a second measurement unit in Embodiment 2 of the present invention.

FIG. 16 is a block diagram showing a configuration of an ultrasound diagnostic apparatus according to Embodiment 3 of the present invention.

FIG. 17 is a flow chart showing an operation of the ultrasound diagnostic apparatus according to Embodiment 3 of the present invention.

FIG. 18 is a block diagram showing an internal configuration of a second measurement unit in Embodiment 3 of the present invention.

### DESCRIPTION OF THE PREFERRED EMBODIMENTS

Embodiments of the invention will be described below based on the appended drawings.

The following description of the constituent elements may be made on the basis of representative embodiments of the present invention, but the present invention is not limited to such embodiments.

In the present specification, the numerical range represented by “to” means a range including numerical values denoted before and after “to” as a lower limit value and an upper limit value.

In the present specification, the term “identical” or “same” means to include an error range generally allowable in the technical field.

### Embodiment 1

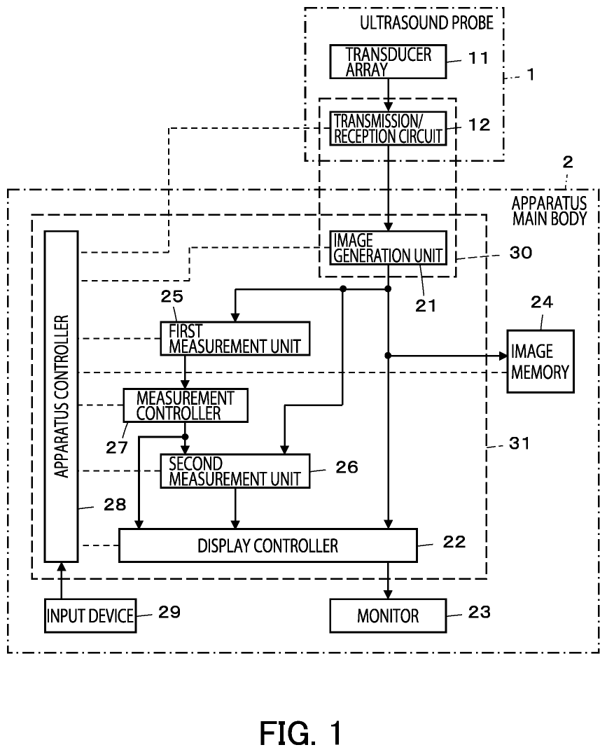

FIG. 1 shows a configuration of an ultrasound diagnostic apparatus according to Embodiment 1 of the present invention. The ultrasound diagnostic apparatus comprises an ultrasound probe 1 and an apparatus main body 2 connected to each other by so-called wired communication or so-called wireless communication.

The ultrasound probe 1 comprises a transducer array 11 and a transmission/reception circuit 12 connected to the transducer array 11.

The apparatus main body 2 comprises an image generation unit 21 connected to the transmission/reception circuit 12. In the apparatus main body 2, a display controller 22 and a monitor 23 are sequentially connected to the image generation unit 21. An image memory 24, a first measurement unit 25, and a second measurement unit 26 are connected to the image generation unit 21. The second measurement unit 26 is connected to the display controller 22. In addition, a measurement controller 27 is connected to the first measurement unit 25. The measurement controller 27 is connected to the second measurement unit 26 and the display controller 22. Further, an apparatus controller 28 is connected to the transmission/reception circuit 12, the image generation unit 21, the image memory 24, the first measurement unit 25, the second measurement unit 26, and the measurement controller 27. An input device 29 is connected to the apparatus controller 28.

The transmission/reception circuit 12 and the image generation unit 21 constitute an image acquisition unit 30. In addition, the image generation unit 21, the display controller 22, the first measurement unit 25, the second measurement unit 26, the measurement controller 27, and the apparatus controller 28 constitute a processor 31 for the apparatus main body 2.

The transducer array 11 of the ultrasound probe 1 has a plurality of ultrasound transducers arranged one-dimensionally or two-dimensionally. Each of these ultrasound transducers transmits an ultrasound wave in accordance with a drive signal supplied from the transmission/reception circuit 12 and receives an ultrasound echo from a subject to output a signal based on the ultrasound echo. Each ultrasound transducer is configured by forming electrodes at both ends of a piezoelectric body consisting of, for example, a piezoelectric ceramic represented by lead zirconate titanate (PZT), a polymer piezoelectric element represented by poly vinylidene di fluoride (PVDF), a piezoelectric single crystal represented by lead magnesium niobate-lead titanate (PMN-PT), or the like.

The image acquisition unit 30 constituted of the transmission/reception circuit 12 and the image generation unit 21 acquires a plurality of frames of ultrasound images in which a subject's heart is imaged as a moving image by transmitting and receiving ultrasound beams using the ultrasound probe 1.

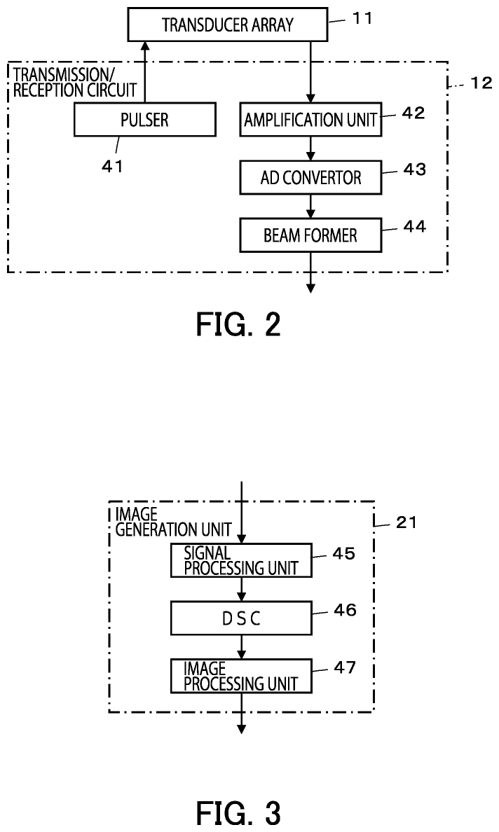

The transmission/reception circuit 12 transmits the ultrasound waves from the transducer array 11 and generates a sound ray signal based on reception signals acquired by the transducer array 11 under control of the apparatus controller 28. As shown in FIG. 2, the transmission/reception circuit 12 includes a pulser 41 connected to the transducer array 11, and an amplification unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus controller 28 so that the ultrasound waves transmitted from the plurality of ultrasound transducers of the transducer array 11 form ultrasound beams, and supplies each drive signal to the plurality of ultrasound transducers. As described above, in a case where a pulsed or continuous wave-like voltage is applied to electrodes of the ultrasound transducer of the transducer array 11, the piezoelectric body expands and contracts to generate pulsed or continuous wave-like ultrasound waves from each of the ultrasound transducers, whereby the ultrasound beam is formed from a combined wave of the ultrasound waves.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In this case, each of the ultrasound transducers constituting the transducer array 11 expands and contracts by receiving the propagating ultrasound echo to generate a reception signal, which is an electrical signal, and outputs these reception signals to the amplification unit 42.

The amplification unit 42 amplifies the signal input from each of the ultrasound transducers constituting the transducer array 11 and transmits the amplified signal to the AD conversion unit 43. The AD conversion unit 43 converts the signal transmitted from the amplification unit 42 into digital reception data. The beam former 44 performs so-called reception focus processing of applying delays to respective pieces of the reception data received from the AD conversion unit 43 and adding up the results. Through this reception focus processing, a sound ray signal in which each piece of the reception data converted by the AD conversion unit 43 is phase-added and the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 corrects attenuation caused by distance of the sound ray signal received from the transmission/reception circuit 12 in accordance with depths of reflection positions of the ultrasound waves using a sound speed value set by the apparatus controller 28 and then performs envelope detection processing on the sound ray signal to generate a B-mode image signal that is tomographic image information related to tissues in the subject.

The DSC 46 converts (raster-converts) the B-mode image signal, which is generated by the signal processing unit 45, into an image signal in accordance with a normal television signal scanning method.

The image processing unit 47 performs various types of necessary image processing such as gradation processing on the B-mode image signal input from the DSC 46, and then transmits the B-mode image signal to the display controller 22, the image memory 24, the first measurement unit 25, and the second measurement unit 26. Hereinafter, the B-mode image signal that has been subjected to image processing by the image processing unit 47 will be referred to as an ultrasound image.

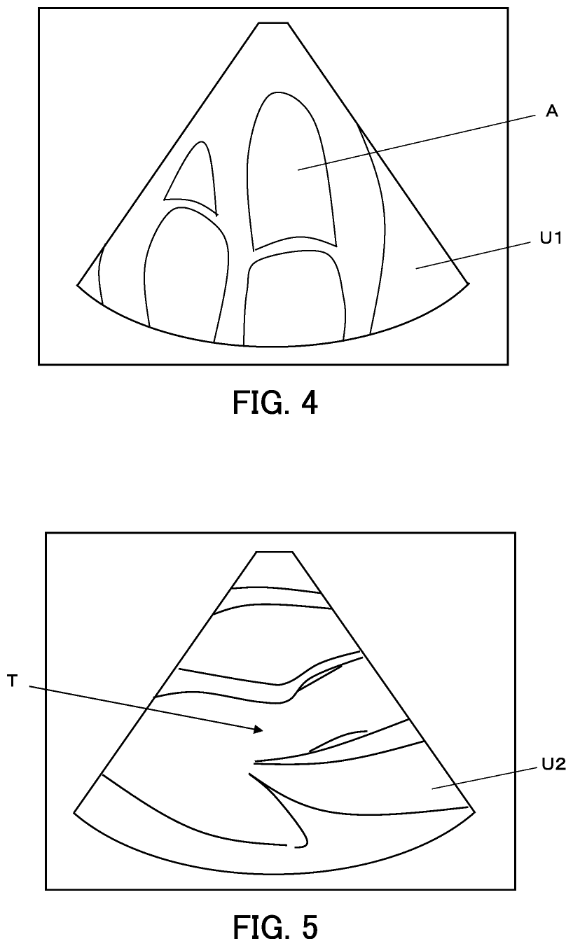

In the present invention, the image acquisition unit 30 acquires the ultrasound images, in each of which the subject's heart is imaged. For measurement of a so-called left ventricular ejection fraction, the image acquisition unit 30 can acquire an ultrasound image U1 showing a so-called apical four-chamber view as schematically shown in FIG. 4, for example. The apical four-chamber view is a cross section including four inner cavities, i.e., a left ventricle A, a left atrium, a right ventricle, and a right atrium.

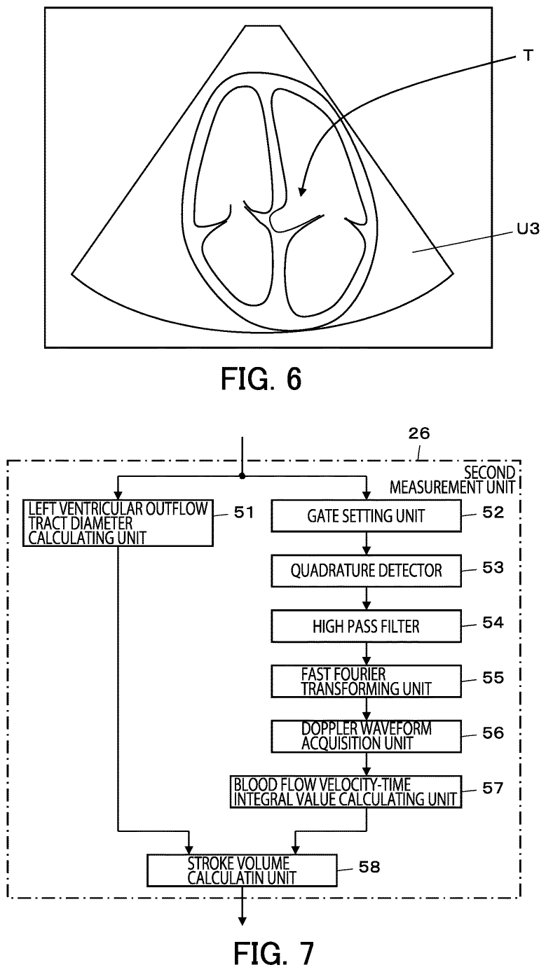

For measurement of a so-called stroke volume or a so-called cardiac output, the image acquisition unit 30 can acquire an ultrasound image U2 showing a so-called parasternal left ventricular long-axis view as schematically shown in FIG. 5, for example. The parasternal left ventricular long-axis view is a cross section including a left ventricle, a left ventricular outflow tract T, a left atrium, and a right ventricle. For measurement of the stroke volume or the cardiac output, the image acquisition unit 30 can also acquire an ultrasound image U3 showing a so-called apical five-chamber view as schematically shown in FIG. 6, for example. The apical five-chamber view is a cross section of a heart including five inner cavities, i.e., a left ventricle, a left ventricular outflow tract T, a left atrium, a right ventricle, and a right atrium.

For measurement of the left ventricular ejection fraction, the image acquisition unit 30 can acquire, in place of the ultrasound image U1 showing an apical four-chamber view, an ultrasound image showing, for example, a so-called apical two-chamber view, apical three-chamber view, apical five-chamber view, long-axis view, left sternal border left ventricular long-axis view, left sternal border left ventricular short-axis view, left sternal border long-axis view, left sternal border short-axis view, left sternal border four-chamber view, subcostal four-chamber view, or subcostal short-axis view. In addition, for measurement of the stroke volume or the cardiac output, the image acquisition unit 30 can acquire, in place of the ultrasound image U3 showing an apical five-chamber view, an ultrasound image showing an apical three-chamber view (not shown).

The image memory 24 is a memory for storing a plurality of frames of ultrasound images acquired by the image acquisition unit 30. For instance, a user can use the frames of the ultrasound images stored in the image memory 24 for checking after the examination or other purposes. As the image memory 24, use can be made of, for example, a recording media such as a flash memory, a hard disc drive (HDD), a solid state drive (SSD), a flexible disk (FD), a magneto-optical (MO) disk, a magnetic tape (MT), a random access memory (RAM), a compact disc (CD), a digital versatile disc (DVD), a secure digital (SD) card, or a universal serial bus (USB) memory.

The first measurement unit 25 measures the left ventricular ejection fraction based on an ultrasound image in which the subject's heart is imaged such as the ultrasound image U1 showing an apical four-chamber view. The first measurement unit 25 can recognize the left ventricle A from, for example, a plurality of frames of the ultrasound images U1 which represent the apical four-chamber view shown in FIG. 4 for a period equal to or longer than a heartbeat interval, calculate an area of the recognized left ventricle A, and calculate the left ventricular ejection fraction from the largest value and the smallest value of the area of the left ventricle A within a heartbeat interval.

The first measurement unit 25 can, for example, store a typical image pattern of the left ventricle A in the ultrasound image U1 showing an apical four-chamber view and recognize the left ventricle A in the ultrasound image U1 by a so-called template matching method, i.e., searching within the ultrasound image U1 using this image pattern. In addition, for example, by inputting the ultrasound image U1 into a trained model which has been preliminarily trained through machine learning with a large number of ultrasound images U1 and the left ventricle A that is imaged therein, the first measurement unit 25 can output an image region showing the left ventricle A in the ultrasound image U1.

By measuring the number of pixels of the left ventricle A recognized in the ultrasound image U1, for example, the first measurement unit 25 can calculate an area of the left ventricle A in the ultrasound image U1. The area of the left ventricle A thus calculated changes periodically in accordance with the heartbeat. The area of the left ventricle A ideally becomes largest at so-called end-diastole of a heart and ideally becomes smallest at so-called end-systole of a heart. With the largest value of the area of the left ventricle A at end-diastole being defined as LVEDV and the smallest value of the area of the left ventricle A at end-systole as LVESV, the first measurement unit 25 can calculate the left ventricular ejection fraction LVEF by Equation (1) below.

$$LVEF = \frac{LVEDV - LVESV}{LVEDV} \times 1 \; 00 ( \% ) \qquad ( 1 )$$

The second measurement unit 26 measures a blood flow-related index related to blood flow based on an ultrasound image in which the subject's heart is imaged. The second measurement unit 26 can measure, as the blood flow-related index, a blood flow velocity-time integral value, i.e., a value obtained by integrating the blood flow velocity over a heartbeat interval. The second measurement unit 26 can also measure the left ventricular outflow tract diameter, i.e., a diameter of the left ventricular outflow tract T and calculate the so-called stroke volume as the blood flow-related index, based on the left ventricular outflow tract diameter and the blood flow velocity-time integral value.

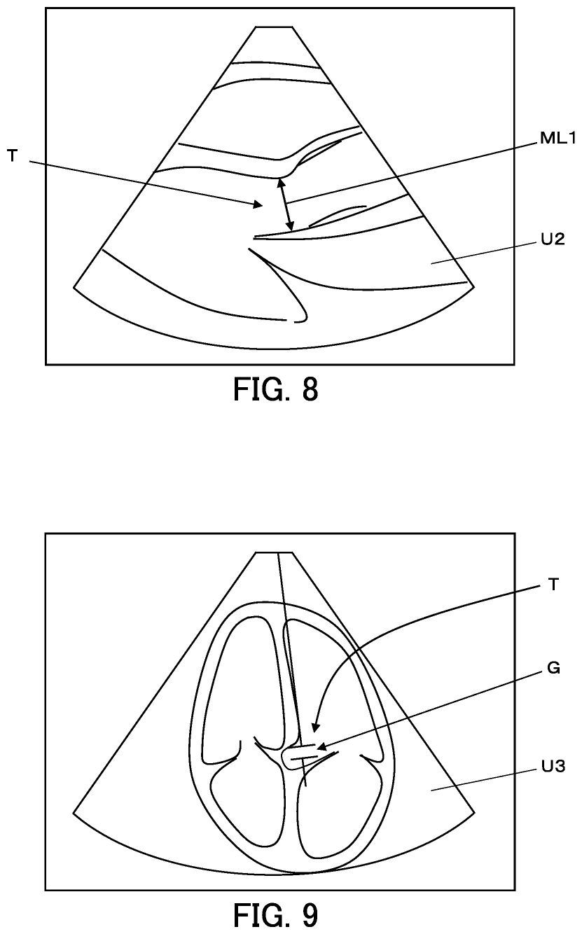

The second measurement unit 26 can have such a configuration as shown in FIG. 7, for example. In the example shown in FIG. 7, the second measurement unit 26 comprises a left ventricular outflow tract diameter measurement unit 51, a gate setting unit 52, a quadrature detector 53, a high pass filter 54, a fast Fourier transforming unit 55, a Doppler waveform acquisition unit 56, a blood flow velocity-time integral value calculating unit 57, and a stroke volume calculating unit 58. The quadrature detector 53, the high pass filter 54, the fast Fourier transforming unit 55, the Doppler waveform acquisition unit 56, and the blood flow velocity-time integral value calculating unit 57 are sequentially connected to the gate setting unit 52. In addition, the stroke volume calculating unit 58 is connected to the left ventricular outflow tract diameter measurement unit 51 and the blood flow velocity-time integral value calculating unit 57.

The left ventricular outflow tract diameter measurement unit 51 can dispose a measurement line ML1 at the left ventricular outflow tract T in the ultrasound image U2 and the like showing the parasternal left ventricular long-axis view as shown in FIG. 8, for example, and measure a length of the disposed measurement line ML1 as the left ventricular outflow tract diameter. The measurement line ML1 can be disposed by the user via the input device 29, for example. The left ventricular outflow tract diameter measurement unit 51 can also automatically dispose the measurement line ML1 based on a position of a local anatomical structure such as an aortic valve in the ultrasound image U2. In this process, the left ventricular outflow tract diameter measurement unit 51 can, for example, recognize the aortic valve and the left ventricular outflow tract in the ultrasound image U2 and dispose the measurement line ML1 at a position within a predetermined distance, e.g., 1 cm from the aortic valve toward the left ventricle A. The left ventricular outflow tract diameter measurement unit 51 can recognize a local anatomical structure in the ultrasound image U2 by the template matching method or a method using a trained model obtained through machine learning, for example.

The gate setting unit 52 sets a so-called Doppler gate G at the left ventricular outflow tract T in the ultrasound image U3 and the like showing an apical five-chamber view as shown in FIG. 9, for example. The Doppler gate G is used to measure the blood flow velocity at the Doppler gate G by the so-called pulse Doppler method. The gate setting unit 52 also can set the Doppler gate G at a position designated by the user via the input device 29, for example. In addition, for example, the gate setting unit 52 can detect the left ventricular outflow tract T by the template matching method or a method using a trained model obtained through machine learning and automatically set the Doppler gate G at the detected left ventricular outflow tract T.

The quadrature detector 53 mixes a sound ray signal received from the transmission/reception circuit 12 with a carrier signal of a reference frequency to thereby convert the sound ray signal into a complex signal through the quadrature detection.

The high pass filter 54 functions as a so-called wall filter and removes a frequency component derived from motion of a tissue in the subject's body from the complex signal generated by the quadrature detector 53.

The fast Fourier transforming unit 55 performs frequency analysis on the complex signal including a plurality of sample points through Fourier transformation, thereby determining the blood flow velocity and generating a spectral signal.

The Doppler waveform acquisition unit 56 aligns the spectral signals generated by the fast Fourier transforming unit 55 on the time axis and represents a magnitude of each frequency component as brightness, thereby generating a Doppler waveform image signal. The Doppler waveform image signal is called a Doppler image, in which a horizontal axis represents the time axis, a vertical axis represents a Doppler shift frequency, i.e., flow velocity, and brightness of a waveform represents power of each frequency component.

The blood flow velocity-time integral value calculating unit 57 calculates a blood flow velocity-time integral (VTI) value based on the Doppler waveform obtained by the Doppler waveform acquisition unit 56. The blood flow velocity-time integral value calculating unit 57 calculates the blood flow velocity-time integral value by identifying a heartbeat interval in the Doppler waveform and time-integrating the value of the flow velocity in the Doppler waveform over the heartbeat interval. By analyzing the Doppler waveform, for example, the blood flow velocity-time integral value calculating unit 57 can identify the waveform repeating unit in the Doppler waveform and identify a period into which the identified repeating unit fits as the heartbeat interval.

The stroke volume calculating unit 58 calculates the stroke volume based on the left ventricular outflow tract diameter measured by the left ventricular outflow tract diameter measurement unit 51 and the blood flow velocity-time integral value calculated by the blood flow velocity-time integral value calculating unit 57. Specifically, assuming that the cross section of the left ventricular outflow tract is circular, the stroke volume calculating unit 58 can calculate a cross sectional area of the left ventricular outflow tract from the left ventricular outflow tract diameter and calculate the stroke volume from a product of the thus calculated cross sectional area and the blood flow velocity-time integral value.

Calculation of a blood flow measurement value by the quadrature detector 53, the high pass filter 54, the fast Fourier transforming unit 55, the Doppler waveform acquisition unit 56, and the blood flow velocity-time integral value calculating unit 57 is performed after a drive signal for transmitting a pulsed ultrasound wave is transmitted to the transducer array 11 under control of the transmission/reception circuit 12 and the apparatus controller 28.

The left ventricular ejection fraction and the blood flow-related index are each used as an indicator in an examination of the cardiac function of the subject. In the meantime, even if the left ventricular ejection fraction shows a normal value, the actual blood delivery amount may be insufficient when an excessively low blood volume results from severe bleeding or dehydration, or the vascular resistance is decreased due to septic shock or the like. Meanwhile, even if the left ventricular ejection fraction is small, it may be possible to measure a sufficient stroke volume or cardiac output by use of preload enhancement or a vasodilator. In addition, even if the left ventricular ejection fraction is determined to fall within a normal value range, a peripheral circulation disorder may be caused in a state where the total blood amount is insufficient due to a low blood volume shock and the like or in a state where a vascular bed is significantly expanded due to sepsis and the like. In a case where the myocardial motion has an abnormality, when mitral regurgitant, aortic valvular stenosis, or another valvular disease is present, a hemodynamic problem which cannot be found from the left ventricular ejection fraction may have arisen. As described above, a disease state regarding the cardiac function cannot be fully found only from the left ventricular ejection fraction in some cases, and in such cases, it may be necessary to acquire a blood flow-related index.

The measurement controller 27 controls the first measurement unit 25 and the second measurement unit 26 such that when the left ventricular ejection fraction measured by the first measurement unit 25 is equal to or smaller than a predetermined threshold value, the second measurement unit 26 measures a blood flow-related index, i.e., a first measurement mode where the first measurement unit 25 measures the left ventricular ejection fraction is automatically shifted to a second measurement mode where the second measurement unit 26 measures a blood flow-related index.

The measurement controller 27 can have, for example, a predetermined threshold value that is set in accordance with the past statistical data as the threshold value for the left ventricular ejection fraction. The threshold value can be set based on, for example, the relationship between the left ventricular ejection fraction obtained from past examinations of a large number of subjects, clinical studies or the like and the outcomes including mortality of the subjects, difficulty in maintaining an effective stroke volume or cardiac output, or frequency of low organ perfusion. For instance, when the left ventricular ejection fraction of smaller than 40% indicates an explicit decrease in the contractile performance, 40% can be set as the threshold value for the left ventricular ejection fraction.

The measurement controller 27 can set the threshold value for the left ventricular ejection fraction to 50% or the like based on, for example, classifications presented in “Guidelines for Diagnosis and Treatment of Acute and Chronic Heart Failure, JCS 2017/JHFS 2017” developed by The Japanese Circulation Society or other guidelines.

The measurement controller 27 can have, for example, a predetermined threshold value that is individually set for each subject as the threshold value for the left ventricular ejection fraction. The threshold value can be set, for example, to a value obtained by subtracting a predetermined value, e.g., 10% from a normal value of the left ventricular ejection fraction of a specific subject.

The measurement controller 27 can have, for example, a predetermined threshold value that is dynamically set based on an evaluation result of fluctuation in at least one of blood pressure, heart rate, or peripheral perfusion index of the subject as the threshold value for the left ventricular ejection fraction. The threshold value can be set, for example, to a value obtained by adding a predetermined value, e.g., 10% when at least one of blood pressure, heart rate, or peripheral perfusion index of the subject fluctuates. The measurement controller 27 can also set, for example, a new threshold value by adding a sufficiently large value to an existing threshold value so that the measurement can be instantly shifted to the measurement of the blood flow-related index by the second measurement unit 26 when at least one of blood pressure, heart rate, or peripheral perfusion index of the subject fluctuates.

As described above, since the measurement controller 27 controls the first measurement unit 25 and the second measurement unit 26 such that when the left ventricular ejection fraction measured by the first measurement unit 25 is equal to or smaller than the predetermined threshold value, the second measurement unit 26 measures the blood flow-related index, even a user with low skills in the cardiac function examination, for example, can instantly move to the measurement of the blood flow-related index with no need for determination of whether the measurement of the blood flow-related index is required. In addition, the user can smoothly proceed with the examination since no manual operation is required when the first measurement mode for measuring the left ventricular ejection fraction is shifted to the second measurement mode for measuring the blood flow-related index.

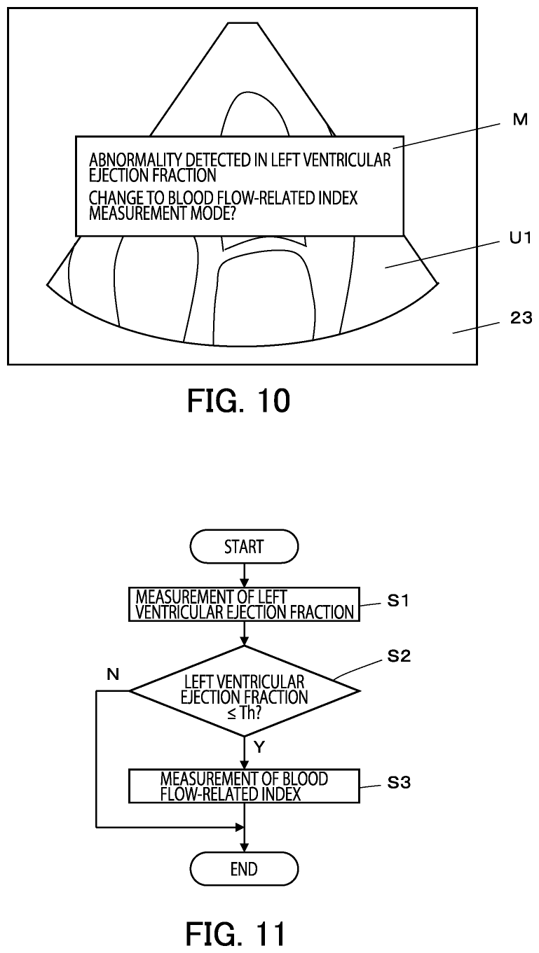

The measurement controller 27 can also cause the monitor 23 to display, for example, a message M as shown in FIG. 10 when the left ventricular ejection fraction measured by the first measurement unit 25 is equal to or smaller than the predetermined threshold value. In this case, for example, the user inputs an instruction for starting the measurement of the blood flow-related index via the input device 29, whereby the measurement is shifted to the second measurement mode for measuring the blood flow-related index. As a result, the user can easily understand that the left ventricular ejection fraction has an abnormality and that the measurement of the blood flow-related index is required to find the disease state of the cardiac function. Meanwhile, in a case where the ultrasound diagnostic apparatus comprises a speaker that is not shown, the measurement controller 27 can deliver a message to the user via the speaker.

The display controller 22 performs predetermined processing on the ultrasound image acquired by the image acquisition unit 30 under control of the apparatus controller 28, the message M issued by the measurement controller 27, and the like, and displays them on the monitor 23.

The monitor 23 is used to display the ultrasound image acquired by the image acquisition unit 30 under control of the display controller 22, the message M issued by the measurement controller 27, and the like, and includes, for example, a display device such as a liquid crystal display (LCD) or an organic electroluminescence display (organic EL display).

The input device 29 is a device for the user to perform an input operation and includes, for example, a device such as a keyboard, a mouse, a track ball, a touch pad, and a touch sensor disposed on the monitor 23 in a superimposed manner.

In the present embodiment, each process in the processor 31 is executed by any computer. In addition, any computer may execute these processes using the processor as hardware, a program as software, or a combination thereof. In that case, the processor is configured to execute various processes in the present embodiment in cooperation with the program, and can function as each unit or each means in the present embodiment. In addition, the order in which the processes are executed by the processor is not limited to the order described above and may be changed as appropriate. Any computer may be a general-purpose computer, a computer for a specific use, a workstation, or another system capable of executing each process.

The processor 31 may be constituted of one or more pieces of hardware, and the type of hardware is not limited. For example, the processor 31 may be constituted of a programmable logic device such as a central processing unit (CPU), a micro processing unit (MPU), or a field programmable gate array (FPGA), a dedicated circuit for executing specific processing such as an application specific integrated circuit (ASIC), or hardware such as a graphics processing unit (GPU) or a neural processing unit (NPU). In addition, the types of hardware may be a combination of different types of hardware. In a case where a plurality of pieces of hardware are configured to execute one or a plurality of processes of a processor, the plurality of pieces of hardware may be present in devices physically separated from each other, or may be present in the same device. In addition, in any of the embodiments, the order of the processes executed by the processor 31 is not limited to the above order and may be changed as appropriate. The hardware is constituted of an electric circuit (circuitry) in which circuit elements such as semiconductor elements are combined.

Further, the program may be software such as firmware or a microcode. In addition, the program may be, for example, a program module group, and each function thereof may be realized by the processor configured to execute each function. The program may be a program code or a plurality of code segments stored in one or a plurality of non-transitory computer-readable media (for example, a storage medium or other storage). The program may be divided and stored in a plurality of non-transitory computer-readable media present in devices physically separated from each other. The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

Next, an operation of the ultrasound diagnostic apparatus according to Embodiment 1 will be described with reference to a flowchart shown in FIG. 11. It should be noted that the sign “Th” in Step S2 in FIG. 11 means a threshold value set to the left ventricular ejection fraction.

In Step S1, the first measurement unit 25 measures the left ventricular ejection fraction based on, for example, the ultrasound image U1 showing an apical four-chamber view. The first measurement unit 25 can recognize the left ventricle A from a plurality of frames of the ultrasound images U1 which represent the apical four-chamber view shown in FIG. 4, for example, in a period equal to or longer than a heartbeat interval, calculate the area of the recognized left ventricle A, and calculate the left ventricular ejection fraction by Equation (1) using the largest value and the smallest value of the area of the left ventricle A within a heartbeat interval.

In Step S2, the measurement controller 27 determines whether the left ventricular ejection fraction measured in Step S1 is equal to or smaller than the predetermined threshold value Th. The measurement controller 27 can have, as the predetermined threshold value Th of the left ventricular ejection fraction, for example, a threshold value that is set based on the past statistical data, a threshold value that is set based on a classification presented in guidelines, a threshold value that is separately set depending on the subject, or a threshold value that is dynamically set based on an evaluation result of fluctuation in at least one of the blood pressure, heart rate, or peripheral perfusion index of the subject.

In a case where the left ventricular ejection fraction is determined to be larger than the predetermined threshold value Th in Step S2, with a judgment that it is sufficiently possible to understand the cardiac function of the subject based on the left ventricular ejection fraction, Step S3 is skipped, and the operation of the ultrasound diagnostic apparatus according to the flowchart of FIG. 11 can be completed. In a case where the left ventricular ejection fraction is determined to be equal to or smaller than the predetermined threshold value Th in Step S2, the operation proceeds to Step S3. At this time, as shown in FIG. 10 for example, the measurement controller 27 can cause the monitor 23 to display the message M indicating that abnormality is detected in the left ventricular ejection fraction and the measurement is shifted to the second measurement mode for measuring the blood flow-related index.

As described above, since the first measurement unit 25 and the second measurement unit 26 are controlled such that when the left ventricular ejection fraction measured by the first measurement unit 25 is equal to or smaller than the predetermined threshold value, the second measurement unit 26 measures the blood flow-related index, even a user with low skills in the cardiac function examination, for example, can instantly move to the measurement of the blood flow-related index with no need for determination of whether the measurement of the blood flow-related index is required. In addition, the user can smoothly proceed with the examination since no manual operation is required when the first measurement mode for measuring the left ventricular ejection fraction is shifted to the second measurement mode for measuring the blood flow-related index.

In Step S3, the second measurement unit 26 measures the blood flow-related index related to the blood flow based on the ultrasound image U2 showing the parasternal left ventricular long-axis view, the ultrasound image U3 showing the apical five-chamber view, or another ultrasound image in which the subject's heart is imaged. The second measurement unit 26 can measure the blood flow velocity-time integral value or the stroke volume as the blood flow-related index.

In this process, the second measurement unit 26 can dispose the measurement line ML1 at the left ventricular outflow tract T in the ultrasound image U2 showing the parasternal left ventricular long-axis view as shown in FIG. 8, for example, and measure a length of the disposed measurement line ML1 as the left ventricular outflow tract diameter.

In addition, as shown in FIG. 9, the second measurement unit 26 sets a so-called Doppler gate G at the left ventricular outflow tract T in, for example, the ultrasound image U3 showing the apical five-chamber view or the like and mixes a sound ray signal received from the transmission/reception circuit 12 based on an ultrasound echo from the position in the subject corresponding to the Doppler gate G with a carrier signal of a reference frequency to thereby convert the sound ray signal into a complex signal through quadrature detection. Further, the second measurement unit 26 removes a frequency component derived from motion of a tissue in the subject's body from the complex signal, performs frequency analysis on the complex signals of a plurality of sample points through Fourier transformation to determine the blood flow velocity and generate spectral signals, acquires a Doppler waveform image signal by representing a magnitude of each frequency component as brightness with the spectral signals being aligned on the time axis, and calculates the blood flow velocity-time integral value based on the Doppler waveform. The second measurement unit 26 can also calculate the stroke volume based on the measured left ventricular outflow tract diameter and the calculated blood flow velocity-time integral value.

The user such as a doctor can find the disease state regarding the subject's cardiac function in more detail by checking the blood flow-related index calculated in Step S3 than by checking the left ventricular outflow tract. When the process of Step S3 is completed in this manner, the operation of the ultrasound diagnostic apparatus according to the flowchart shown in FIG. 11 is completed.

As described above, with the ultrasound diagnostic apparatus in Embodiment 1 of the present invention, the first measurement unit 25 measures the left ventricular ejection fraction based on the ultrasound image in which the subject's heart is imaged, the second measurement unit 26 measures the blood flow-related index related to blood flow based on the ultrasound image, and the measurement controller 27 controls the first measurement unit 25 and the second measurement unit 26 such that when the left ventricular ejection fraction measured by the first measurement unit 25 is equal to or smaller than the predetermined threshold value, the second measurement unit 26 measures the blood flow-related index. Therefore, it is possible to save the user's workload to change from the first measurement mode for measuring the left ventricular outflow tract to the second measurement mode for measuring the blood flow-related index and smoothly carry out the examination of the subject's cardiac function.

A case has been described in which the transmission/reception circuit 12 is provided in the ultrasound probe 1, but the transmission/reception circuit 12 may be provided in the apparatus main body 2.

In addition, a case has been described in which the image generation unit 21 is provided in the apparatus main body 2, but the image generation unit 21 may be provided in the ultrasound probe 1.

The apparatus main body 2 may be a so-called stationary type, a portable type that is easily carried, or a so-called handheld type that is constituted of, for example, a smartphone or a tablet type computer. In this way, the type of the device constituting the apparatus main body 2 is not particularly limited.

A case has been described in which the stroke volume is calculated based on the left ventricular outflow tract measured by the left ventricular outflow tract diameter measurement unit 51 and the blood flow velocity-time integral value calculated by the blood flow velocity-time integral value calculating unit 57, but when the left ventricular outflow tract diameter has been accurately measured in the subject's past examination, the second measurement unit 26 can calculate the stroke volume using the left ventricular outflow tract diameter measured in the past in place of the left ventricular outflow tract diameter measured in the present examination. In this case, the user preliminarily inputs an instruction for calculating the stroke volume using the past left ventricular outflow tract diameter via the input device 20, and the second measurement unit 26 can calculate the stroke volume using the left ventricular outflow tract diameter measured in the past in accordance with the instruction input by the user.

Moreover, when the left ventricular outflow tract diameter does not largely fluctuate in plural past examinations of the subject and the subject is not found to have a disease e.g., valvular disease or aortic valvular stenosis contributing to a change in the left ventricular outflow tract, for example, the second measurement unit 26 can calculate the blood flow velocity-time integral value as the blood flow-related index while omitting the measurement of the left ventricular outflow tract diameter. In this case, the user inputs an instruction for omitting the measurement of the left ventricular outflow tract diameter via the input device 20, and the second measurement unit 26 can calculate the blood flow velocity-time integral value as the blood flow-related index in accordance with the instruction input by the user.

Also when it is difficult to measure the left ventricular outflow tract diameter due to the subject's tolerable posture, the subject's respiratory status, or the like, the second measurement unit 26 can calculate the blood flow velocity-time integral value as the blood flow-related index while omitting the measurement of the left ventricular outflow tract diameter in accordance with the instruction input by the user.

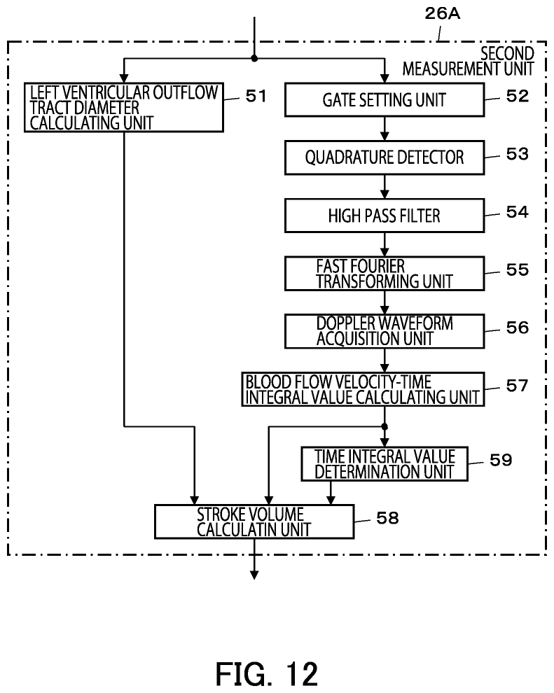

The measurement of the blood flow velocity-time integral value may be performed after the measurement of the left ventricular outflow tract diameter, and the measurement of the left ventricular outflow tract diameter may be performed after the measurement of the blood flow velocity-time integral value. In a case where the measurement of the left ventricular outflow tract diameter is performed after the measurement of the blood flow velocity-time integral value, the second measurement unit 26 determines whether the blood flow velocity-time integral value falls within an acceptable range and can omit the subsequent measurement of the left ventricular outflow tract diameter when the blood flow velocity-time integral value falls within the acceptable range. In this case, the ultrasound diagnostic apparatus can comprise, for example, a second measurement unit 26A shown in FIG. 12 in place of the second measurement unit 26 shown in FIG. 7. The second measurement unit 26A further comprises a time integral value determination unit 59 in the second measurement unit 26 shown in FIG. 7. The time integral value determination unit 59 is connected to the blood flow velocity-time integral value calculating unit 57 in the second measurement unit 26A. The time integral value determination unit 59 is connected to the stroke volume calculating unit 58.

The time integral value determination unit 59 determines whether the measurement result of the blood flow velocity-time integral value obtained by the second measurement unit 26A, i.e., the blood flow velocity-time integral value obtained by the blood flow velocity-time integral value calculating unit 57 falls within a predetermined acceptable range of time integral value or not. Generally, the normal value of the adult's blood flow velocity-time integral value is about 18 cm to 22 cm. Hence, the acceptable range of the time integral value can be set to a range including a normal value of about 18 cm to 22 cm. In addition, based on the blood flow velocity-time integral values obtained in the past plural examinations of the subject, a range including a normal blood flow velocity-time integral value of the subject can be set as the acceptable range of the time integral value.

The second measurement unit 26A can omit the measurement of the left ventricular outflow tract diameter when the time integral value determination unit 59 determines that the blood flow velocity-time integral value falls within the acceptable range of the time integral value. At this time, the second measurement unit 26A can calculate the blood flow velocity-time integral value as the blood flow-related index. The stroke volume calculating unit 58 can also calculate, as the blood flow-related index, the stroke volume based on a specified value of the left ventricular outflow tract diameter such as a left ventricular outflow tract diameter measured in a past examination of the subject, and the blood flow velocity-time integral value calculated by the blood flow velocity-time integral value calculating unit 57.

The second measurement unit 26A can omit the measurement of the left ventricular outflow tract diameter also when the blood flow velocity-time integral value falls within the acceptable range of the time integral value while the blood pressure, the peripheral perfusion condition, urine output, uric acid level, or another clinical condition of the subject can be determined to be improving or to be stable. Information regarding the subject's clinical condition is input by the user via the input device 29, for example.

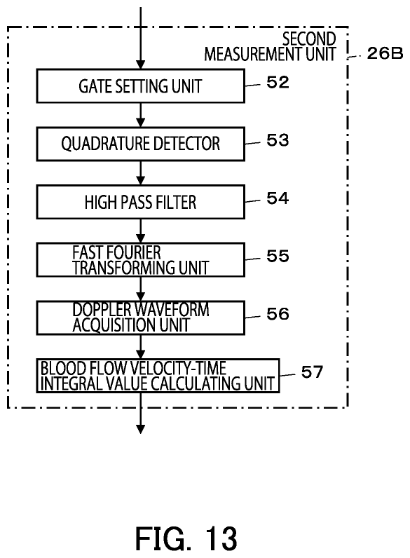

The ultrasound diagnostic apparatus can also comprise, as shown in FIG. 13, a second measurement unit 26B configured by removing the left ventricular outflow tract diameter calculating unit 51 and the stroke volume calculating unit 58 from the second measurement unit 26 shown in FIG. 7 for a case where the blood flow velocity-time integral value is calculated as the blood flow-related index.

The second measurement unit 26 can also calculate a so-called cardiac output as the blood flow-related index. In this case, the second measurement unit 26 can comprise, for example, a heart rate measurement unit and a cardiac output calculating unit that are sequentially connected to the stroke volume calculating unit 58. The heart rate measurement unit can measure the heart rate by, for example, detecting a cycle of the repeating unit of the Doppler waveform acquired by the Doppler waveform acquisition unit 56. The cardiac output calculating unit can calculate the cardiac output from a product of the measured heart rate and the stroke volume.

### Embodiment 2

In a case where the so-called cardiac output is calculated as the blood flow-related index, the cardiac output cannot be accurately calculated in some cases when the heart rate fluctuates. In this case, an ultrasound diagnostic apparatus can notify a user of fluctuation in the heart rate.

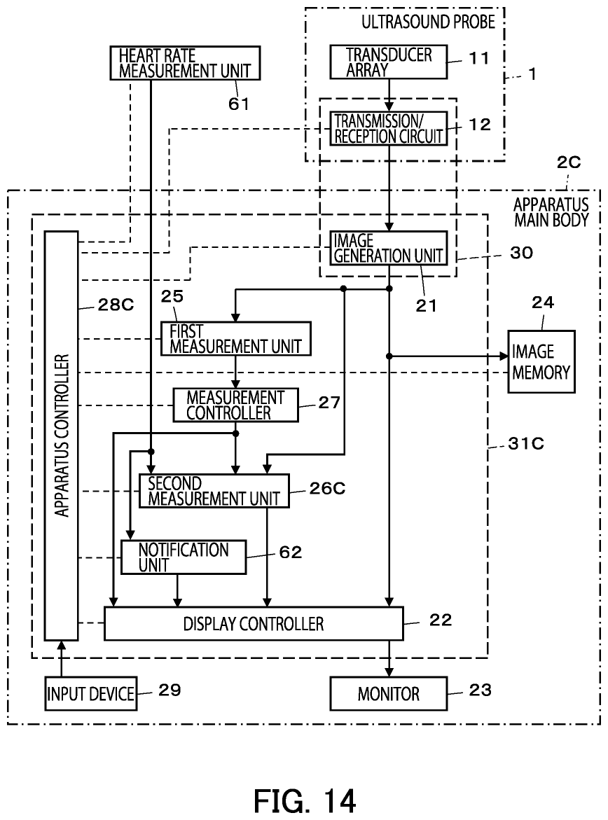

FIG. 14 illustrates a configuration of an ultrasound diagnostic apparatus of Embodiment 2. The ultrasound diagnostic apparatus of Embodiment 2 further comprises a heart rate measurement unit 61 and comprises an apparatus main body 2C in place of the apparatus main body 2 in the ultrasound diagnostic apparatus of Embodiment 1 shown in FIG. 1. The apparatus main body 2C further comprises a notification unit 62 and comprises a second measurement unit 26C and an apparatus controller 28C in place of the second measurement unit 26 and the apparatus controller 28, respectively, in the apparatus main body 2 in Embodiment 1.

To the heart rate measurement unit 61, the second measurement unit 26C and the display controller 22 are connected. The second measurement unit 26C is connected to the measurement controller 27 and the display controller 22. The image generation unit 21, the display controller 22, the first measurement unit 25, the second measurement unit 26C, the measurement controller 27, the display controller 28C, and the notification unit 62 constitute a processor 31C for the apparatus main body 2C.

The heart rate measurement unit 61 is constituted of, for example, a device for measuring a heart rate such as a so-called electrocardiograph that is attached to the subject, and measures the subject's heart rate.

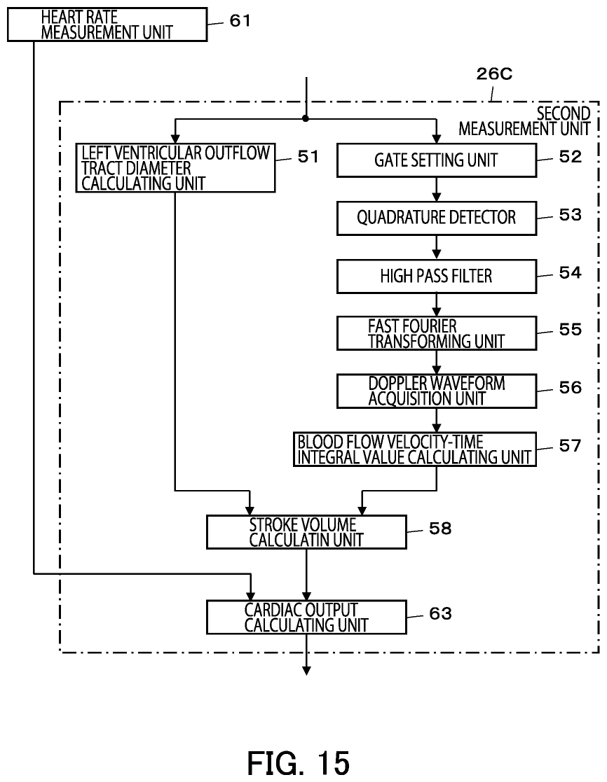

The second measurement unit 26C calculates the cardiac output based on the stroke volume calculated by the stroke volume calculating unit 58 and the subject's heart rate measured by the heart rate measurement unit 61. The second measurement unit 26C has an internal configuration as shown in FIG. 15, for example. The second measurement unit 26C further comprises a cardiac output calculating unit 63 connected to the stroke volume calculating unit 58 in the second measurement unit 26 in Embodiment 1 shown in FIG. 7. The cardiac output calculating unit 63 is connected to the heart rate measurement unit 61 besides the stroke volume calculating unit 58.

The cardiac output calculating unit 63 calculates a cardiac output based on a product of the stroke volume calculated by the stroke volume calculating unit 58 and the subject's heart rate measured by the heart rate measurement unit 61.

The notification unit 62 notifies the user when the heart rate measured by the heart rate measurement unit 61 fluctuates beyond a predetermined fluctuation range of the heart rate. The notification unit 62 can notify the user by, for example, displaying a message on the monitor 23. The user can confirm the notification from the notification unit 62 and take an action, e.g., talk to the subject, in order to calm the subject down to stabilize the subject's heart rate.

As described above, with the ultrasound diagnostic apparatus in Embodiment 2 of the present invention, since the notification unit 62 notifies the user when the heart rate measured by the heart rate measurement unit 61 fluctuates beyond the predetermined fluctuation range of the heart rate, the user who has confirmed the notification takes an action to stabilize the subject's heart rate, whereby the cardiac output can be accurately calculated.

In addition, a case has been described in which the subject's heart rate is measured by an electrocardiograph, but, for example, the heart rate can be measured based on the Doppler waveform acquired by the Doppler waveform acquisition unit 56 in the second measurement unit 26C. In a case where the heart rate cannot be accurately measured from the Doppler waveform and the like due to, for example, distortion of the Doppler waveform, the apparatus controller 28 can switch the measuring method for the heart rate to the measuring method by use of an electrocardiograph.

### Embodiment 3

The measurement controller can control the second measurement unit 26 additionally taking into consideration the hemodynamics of the subject.

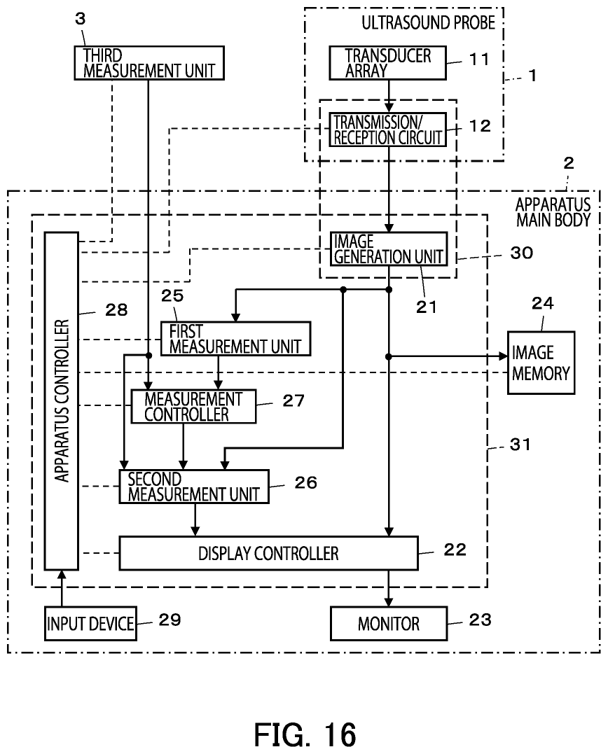

FIG. 16 illustrates a configuration of an ultrasound diagnostic apparatus of Embodiment 3. The ultrasound diagnostic apparatus of Embodiment 3 further comprises a third measurement unit 3 in the ultrasound diagnostic apparatus of Embodiment 1 shown in FIG. 1.

The third measurement unit 3 measures a hemodynamic index representing the hemodynamics of the subject. The third measurement unit 3 can measure, for example, any one of blood pressure, heart rate, peripheral oxygen saturation, lactate level, and peripheral skin temperature as a hemodynamic index. In this case, the third measurement unit 3 can be constituted of, for example, a non-invasive sphygmomanometer for measuring blood pressure as the hemodynamic index, an electrocardiogram monitor for measuring the heart rate as the hemodynamic index, a pulse oximeter for measuring peripheral oxygen saturation as the hemodynamic index, a lactate meter for measuring a lactate level as the hemodynamic index, or a thermometer for measuring peripheral skin temperature as the hemodynamic index.

The measurement controller 27 controls the second measurement unit 26 such that when the left ventricular ejection fraction measured by the first measurement unit 25 is equal to or smaller than the predetermined threshold value and the hemodynamic index measured by the third measurement unit 3 shows abnormality, the blood flow-related index is measured by the second measurement unit 26, that is, the first measurement mode where the left ventricular ejection fraction is measured by the first measurement unit 25 is automatically shifted to the second measurement mode where the blood flow-related index is measured by the second measurement unit 26.

The measurement controller 27 can determine whether the hemodynamics of the subject is abnormal based on a predetermined hemodynamic threshold value with respect to the hemodynamic index of the subject measured by the third measurement unit 3. The hemodynamic threshold value can be set by a facility such as a hospital, or a medical specialty based on, for example, the typical numerical value of the hemodynamic index of the subject. The hemodynamic threshold value can be set also in consideration of the subject information, e.g., the subject's age and underlying disease.

In a case where the hemodynamic index is the systolic blood pressure, the hemodynamic threshold value can be set to 90 mmHg, for example. In a case where the hemodynamic index is the average blood pressure of the subject, the hemodynamic threshold value can be set to 65 mmHg. In a case where the hemodynamic index is blood pressure, the measurement controller 27 can determine that the subject's hemodynamic index is abnormal when the blood pressure measured by the third measurement unit 3 is lower than the hemodynamic threshold value and that the subject's hemodynamic index is normal when the blood pressure is equal to or higher than the hemodynamic threshold value.

In a case where the hemodynamic index is a heart rate at rest, the hemodynamic threshold value can be set to, for example, a value within a range of 100 bpm to 120 bpm. In a case where the hemodynamic index is a heart rate at rest, the measurement controller 27 can determine that the hemodynamic index of the subject is abnormal when the subject's heart rate measured by the third measurement unit 3 is higher than the hemodynamic threshold value and that the hemodynamic index of the subject is normal when the subject's heart rate is equal to or lower than the hemodynamic threshold value.

In a case where the hemodynamic index is peripheral oxygen saturation, the hemodynamic threshold value can be set to a value within a range of 90% to 92%, for example. In a case where the hemodynamic index is peripheral oxygen saturation, the measurement controller 27 can determine that the hemodynamic index of the subject is abnormal when the subject's peripheral oxygen saturation measured by the third measurement unit 3 is equal to or lower the hemodynamic threshold value and that the hemodynamic index of the subject is normal when the subject's peripheral oxygen saturation is higher than the hemodynamic threshold value.

In a case where the hemodynamic index is lactate level, the hemodynamic threshold value can be set to a value within a range of 2.0 mmol/L to 4.0 mmol/L, for example. In a case where the hemodynamic index is lactate level, the measurement controller 27 can determine that the hemodynamic index of the subject is abnormal when the subject's lactate level measured by the third measurement unit 3 is higher than the hemodynamic threshold value and that the hemodynamic index of the subject is normal when the subject's lactate level is equal to or lower than the hemodynamic threshold value.

In a case where the hemodynamic index is peripheral skin temperature, the hemodynamic threshold value can be set to a value within a range of 35.0 °C to 35.5 °C, for example. In a case where the hemodynamic index is peripheral skin temperature, the measurement controller 27 can determine that the hemodynamic index of the subject is abnormal when the subject's peripheral skin temperature measured by the third measurement unit 3 is equal to or lower than the hemodynamic threshold value and that the hemodynamic index of the subject is normal when the subject's peripheral skin temperature is higher than the hemodynamic threshold value.

As described above, since the measurement controller 27 controls the second measurement unit 26 such that when the left ventricular ejection fraction measured by the first measurement unit 25 is equal to or smaller than the predetermined threshold value and the hemodynamics of the subject is determined to be abnormal, the second measurement unit 26 measures the blood flow-related index, even a user with low skills in the cardiac function examination, for example, can instantly move to the measurement of the blood flow-related index with no need for determination of whether the measurement of the blood flow-related measurement is required. In addition, the user can smoothly proceed with the examination since no manual operation is required when the first measurement mode for measuring the left ventricular ejection fraction is shifted to the second measurement mode for measuring the blood flow-related index.

The measurement controller 27 can also cause the monitor 23 to display, for example, the message M as shown in FIG. 10 when the left ventricular ejection fraction measured by the first measurement unit 25 is equal to or smaller than the predetermined threshold value and the subject's hemodynamics is determined to be abnormal. In this case, for example, the user inputs an instruction for starting the measurement of the blood flow-related index via the input device 29, whereby the measurement is shifted to the second measurement mode for measuring the blood flow-related index (blood flow-related index measurement mode). As a result, the user can easily understand that the left ventricular ejection fraction has an abnormality and that the measurement of the blood flow-related index is required to find the disease state of the cardiac function. Meanwhile, in a case where the ultrasound diagnostic apparatus comprises a speaker that is not shown, the measurement controller 27 can deliver a message to the user via the speaker.

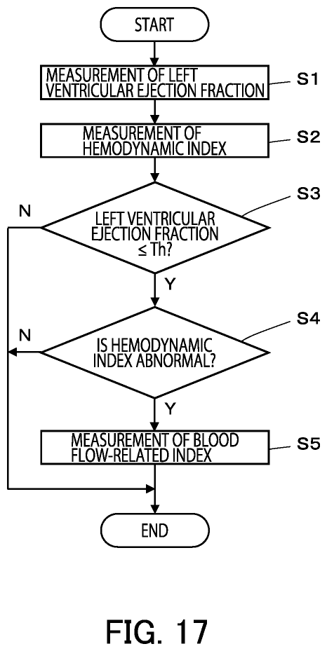

Next, an operation of the ultrasound diagnostic apparatus according to the embodiment will be described with reference to a flowchart shown in FIG. 17. It should be noted that the sign “Th” in Step S2 in FIG. 17 means a predetermined threshold value set to the left ventricular ejection fraction. Step S1, Step S3, and Step S5 in the flowchart of FIG. 17 are the same as Step S1 to Step S3 in the flowchart of FIG. 11, and therefore the detailed description of these steps is omitted.

In Step S1, the first measurement unit 25 measures the left ventricular ejection fraction based on, for example, the ultrasound image U1 showing an apical four-chamber view.

In Step S2, the third measurement unit 3 measures a hemodynamic index representing the hemodynamics of the subject. The third measurement unit 3 can measure any one of blood pressure, heart rate, peripheral oxygen saturation, lactate level, and peripheral skin temperature as the subject's hemodynamic index.

In Step S3, the measurement controller 27 determines whether the left ventricular ejection fraction measured in Step S1 is equal to or smaller than the predetermined threshold value Th.

In a case where the left ventricular ejection fraction is determined to be larger than the predetermined threshold value Th in Step S3, with a judgment that it is sufficiently possible to understand the cardiac function of the subject based on the left ventricular ejection fraction, Step S4 and Step S5 are skipped, and the operation of the ultrasound diagnostic apparatus according to the flowchart of FIG. 11 can be completed. In a case where the left ventricular ejection fraction is determined to be equal to or smaller than the predetermined threshold value Th in Step S3, the operation proceeds to Step S4.

In Step S4, the measurement controller 27 determines whether the hemodynamic index measured in Step S2 shows abnormality. In a case where the hemodynamic index is blood pressure, for example, blood pressure that is lower than the hemodynamic threshold value can be determined to be abnormal, and blood pressure that is equal to or higher than the hemodynamic threshold value can be determined to be normal.

When the hemodynamic index is determined to be normal in Step S4, with a judgment that it is sufficiently possible to understand the cardiac function of the subject based on the left ventricular ejection fraction, Step S5 is skipped, and the operation of the ultrasound diagnostic apparatus according to the flowchart of FIG. 11 can be completed. When the hemodynamic index is determined to be abnormal in Step S4, with a judgment that it is necessary to measure the blood flow-related index, the operation proceeds to Step S5. At this time, as shown in FIG. 10 for example, the measurement controller 27 can cause the monitor 23 to display the message M indicating that abnormality is detected in the hemodynamics of the subject and the measurement is shifted to the second measurement mode for measuring the blood flow-related index.

As described above, since the second measurement unit 26 is controlled such that when the left ventricular ejection fraction measured by the first measurement unit 25 is equal to or smaller than the predetermined threshold value Th in Step S3 and the hemodynamic index is determined to be abnormal in Step S4, the second measurement unit 26 measures the blood flow-related index, even a user with low skills in the cardiac function examination, for example, can instantly move to the measurement of the blood flow-related index with no need for determination of whether the measurement of the blood flow-related measurement is required. In addition, the user can smoothly proceed with the examination since no manual operation is required when the first measurement mode for measuring the left ventricular ejection fraction is shifted to the second measurement mode for measuring the blood flow-related index.

In Step S5, the second measurement unit 26 measures the blood flow-related index related to the blood flow based on the ultrasound image U2 showing the parasternal left ventricular long-axis view, the ultrasound image U3 showing the apical five-chamber view, and another ultrasound image in which the subject's heart is imaged.

The user such as a doctor can find the disease state regarding the subject's cardiac function in more detail by checking the blood flow-related index calculated in Step S5 than by checking the left ventricular outflow tract. When the process of Step S5 is completed in this manner, the operation of the ultrasound diagnostic apparatus according to the flowchart shown in FIG. 17 is completed.

As described above, with the ultrasound diagnostic apparatus in Embodiment 3 of the present invention, the first measurement unit 25 measures the left ventricular ejection fraction based on the ultrasound image in which the subject's heart is imaged, the second measurement unit 26 measures the blood flow-related index related to the blood flow based on the ultrasound image, the third measurement unit 3 measures the hemodynamic index representing hemodynamics of the subject, and the measurement controller 27 controls the second measurement unit 26 such that when the left ventricular ejection fraction is equal to or larger than the predetermined threshold value and the hemodynamic index shows abnormality, the second measurement unit 26 measures the blood flow-related index. Therefore, it is possible to save the user's workload to change from the first measurement mode for measuring the left ventricular outflow tract to the second measurement mode for measuring the blood flow-related index and smoothly carry out the examination of the subject's cardiac function.

While the hemodynamic index is measured after the measurement of the left ventricular ejection fraction in the flowchart of FIG. 17, the ultrasound diagnostic apparatus can measure the left ventricular ejection fraction after the measurement of the hemodynamic index and can measure the left ventricular ejection fraction and the hemodynamic index at the same time.

The second measurement unit 26 can also calculate a so-called cardiac output as the blood flow-related index.

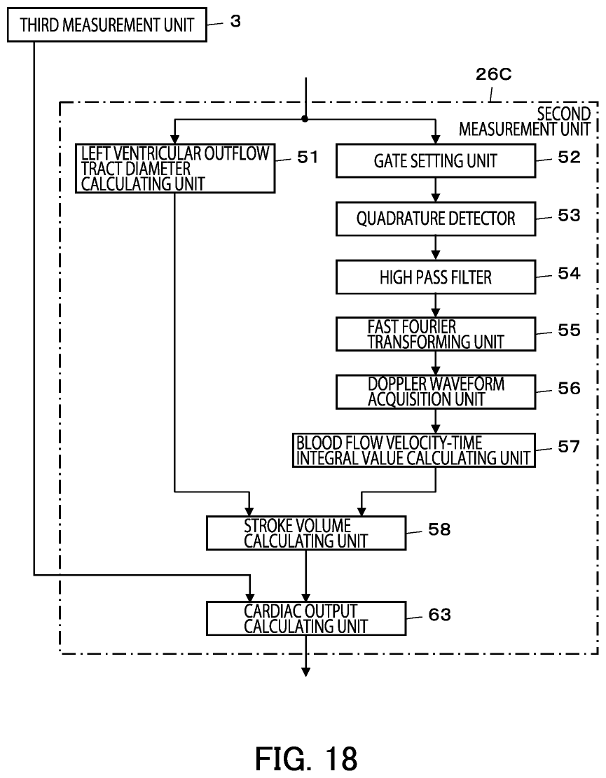

In this case, the ultrasound diagnostic apparatus can comprise, for example, the second measurement unit 26C shown in FIG. 18 as with the second measurement unit 26C shown in FIG. 15, in place of the second measurement unit 26 shown in FIG. 7. The second measurement unit 26C further comprises the cardiac output calculating unit 63 in the second measurement unit 26 shown in FIG. 7. In the second measurement unit 26C, the third measurement unit 3 and the stroke volume calculating unit 58 are connected to the cardiac output calculating unit 63.

### EXPLANATION OF REFERENCES

1: ultrasound probe,
2, 2C: apparatus main body
3: third measurement unit
11: transducer array
12: transmission/reception circuit
21: image generation unit
22: display controller
23: monitor
24: image memory
25: first measurement unit
26, 26A, 26B, 26C: second measurement unit
27: measurement controller
28, 28C: apparatus controller
29: input device
30: image acquisition unit
31, 31C: processor
41: pulser
42: amplification unit
43: AD convertor
44: beam former
45: signal processing unit
46: DSC
47: image processing unit
51: left ventricular outflow tract diameter calculating unit
52: gate setting unit
53: quadrature detector
54: high pass filter
55: fast Fourier transforming unit
56: Doppler waveform acquisition unit
57: blood flow velocity-time integral value calculating unit
58: stroke volume calculating unit
59: time integral value determination unit
61: heart rate measurement unit
62: notification unit
63: cardiac output calculating unit
A: left ventricle
G: Doppler gate
M: message
ML1: measurement line
T: left ventricular outflow tract
U1, U2, U3: ultrasound image

What is claimed is:

1. An ultrasound diagnostic apparatus comprising:
a processor configured to:
measure a left ventricular ejection fraction based on an ultrasound image in which a heart of a subject is imaged;
upon determining that the measured left ventricular ejection fraction is equal to or smaller than a predetermined threshold value, measure a first index value related to blood flow based on the ultrasound image.

2. The ultrasound diagnostic apparatus according to claim 1,
wherein the processor is configured to:
measure a blood flow velocity-time integral value, and
acquire a stroke volume as the first index value based on the measured blood flow velocity-time integral value and a left ventricular outflow tract diameter.

3. The ultrasound diagnostic apparatus according to claim 2,
wherein the processor is configured to:
measure the left ventricular outflow tract diameter, and
acquire the stroke volume based on the measured left ventricular outflow tract diameter and the measured blood flow velocity-time integral value.

4. The ultrasound diagnostic apparatus according to claim 2,
wherein the processor is configured to:
determine whether a measurement result of the blood flow velocity-time integral value falls within a predetermined acceptable range of time integral value; and
upon determining that the blood flow velocity-time integral value falls within the acceptable range of the time integral value, acquire the stroke volume based on a specified value of the left ventricular outflow tract diameter and the measured blood flow velocity-time integral value.

5. The ultrasound diagnostic apparatus according to claim 2,
wherein the processor is configured to acquire a heart rate of the subject.

6. The ultrasound diagnostic apparatus according to claim 5,
wherein the processor is configured to calculate a cardiac output based on the acquired stroke volume and the acquired heart rate.

7. The ultrasound diagnostic apparatus according to claim 5,
wherein the processor is configured to, upon determining that the acquired heart rate fluctuate beyond a predetermined fluctuation range, notify a user.

8. The ultrasound diagnostic apparatus according to claim 1,
wherein the processor is configured to acquire a blood flow velocity-time integral value as the first index value.

9. The ultrasound diagnostic apparatus according to claim 1,
wherein the processor is configured to use the predetermined threshold value that is set based on past statistical data.

10. The ultrasound diagnostic apparatus according to claim 1,
wherein the processor is configured to use the predetermined threshold value that is individually set depending on the subject.

11. The ultrasound diagnostic apparatus according to claim 10,
wherein the processor is configured to dynamically set the predetermined threshold value based on an evaluation result of fluctuation in at least one of blood pressure, heart rate, or peripheral perfusion index of the subject.

12. The ultrasound diagnostic apparatus according to claim 1,
wherein the processor is configured to:
acquire a second index value representing hemodynamics of the subject, and
upon determining that the measured left ventricular ejection fraction measured is equal to or larger than a predetermined threshold value and the second index value shows abnormality, measure the blood flow-related index is measured by the second measurement unit.

13. The ultrasound diagnostic apparatus according to claim 12, further comprising:
a non-invasive sphygmomanometer configured to measure blood pressure,
wherein the processor is configured to acquire the blood pressure as the second index value from the non-invasive sphygmomanometer.

14. The ultrasound diagnostic apparatus according to claim 12, further comprising:
an electrocardiogram monitor configured to measure a heart rate,
wherein the processor is configured to acquire the heart rate as the second index value from the electrocardiogram monitor.

15. The ultrasound diagnostic apparatus according to claim 12, further comprising:
a pulse oximeter configured to measure peripheral oxygen saturation,
wherein the processor is configured to acquire the peripheral oxygen saturation as the second index value from the pulse oximeter.

16. The ultrasound diagnostic apparatus according to claim 12, further comprising:
a lactate meter configured to measure a lactate level,
wherein the processor is configured to acquire the lactate level as the second index value from the lactate meter.

17. The ultrasound diagnostic apparatus according to claim 12, further comprising:
a thermometer configured to measure peripheral skin temperature,
wherein the processor is configured to acquire the peripheral skin temperature as the second index value from the thermometer.

18. The ultrasound diagnostic apparatus according to claim 2, further comprising:
an electrocardiogram monitor configured to measure a heart rate of the subject,
wherein the processor is configured to:
upon determining that the measured left ventricular ejection fraction is equal to or larger than the predetermined threshold value and the measured heart rate shows abnormality,
calculate a cardiac output as the first index value based on the acquired stroke volume and the measured heart rate.

19. A method of controlling an ultrasound diagnostic apparatus, the method comprising:
measuring a left ventricular ejection fraction based on an ultrasound image in which a heart of a subject is imaged; and
upon determining that the measured left ventricular ejection fraction is equal to or smaller than a predetermined threshold value, measuring a first index value related to blood flow based on the ultrasound image.
